(19) United States
(12) Patent Application Publication
(10) Pub. No.: US 2002/0169141 A1
(43) Pub. Date: Nov. 14, 2002

(54) Oral dosage forms for administration of the combination of tegafur, uracil, folinic acid, and irinotecan and method of using the same

(76) Inventors: Christophe Martin, Ophain (BE); Christopher John Twelves, Bearsden Glasgow (GB); Susan Maria Bailey, Cambridge (GB)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 09/971,871

(22) Filed: Oct. 5, 2001

Related U.S. Application Data
(60) Provisional application No. 60/238,650, filed on Oct. 6, 2000.

(51) Int. Cl. A61K 31/7072; A61K 31/525; A61K 31/4745; A61K 31/513

(52) U.S. Cl. 514/50; 514/251; 514/283

(57) ABSTRACT

This invention provides a dosage form and a method of administering an anti-tumor composition comprising tegafur, uracil, and folinic acid to potentiate the coadministration of irinotecan.

### RELATED APPLICATIONS

This application claims the benefit under 35 U.S.C. Section 119(e) of U.S. Provisional Patent Application Ser. No. 60/238,650, filed Oct. 6, 2000.

### FIELD OF THE INVENTION

The present invention is directed to an oral dosage form(s) for administration to a warm blooded animal of the combination of tegafur, uracil, and folinic acid to potentionate coadministered irinotecan for the treatment of tumors.

### BACKGROUND OF THE INVENTION

5-Fluorouracil (5-FU) is known anti-tumor agent. The combination of 5- fluorouracil and folinic acid is a known treatment for colorectal cancer. Tegafur (1- (2-tertrahydrofuryl)-5-fluorouracil) is a prodrug of 5-fluorouracil. In vivo, 5-fluorouracil is rapidly inactivated by the enzyme dihydropyridine dehydrogenase (DPD). Uracil competitively inhibits DPD metabolism of 5-FU generated from tegafur. Thus, coadministration of uracil with tegafur results in higher exposures of active 5-FU as compared to tegafur alone. It is known that 5-fluorouracil cannot be administered orally.

U.S. Pat. No. 4,328,229 discloses an anti-cancer composition containing 1-(2-tetrahydrofuryl)-5-fluorouracil (“tegafur”) and uracil. The composition is used for delivery of 5-fluorouracil to a tumor sensitive to 5- fluorouracil in a warm-blooded animal. It is disclosed that the composition can be administered in a variety of dosage forms including an oral dosage form.

U.S. Pat. No. 5,534,513 discloses an anti-tumor composition containing tegafur and uracil in a molar ratio of 1:4. This anti-tumor composition is stated to be further potentiated by the administration of folinic acid or a pharmaceutically acceptable salt thereof. It is disclosed in the '513 patent that the combination can be administered in a variety of dosage forms including an oral dosage form.

Irinotecan (C₃₃H₃₈N₄O₆) is a known anti-tumor agent as disclosed in T. Kunimoto et al., Cancer Res., Vol. 47, p. 5944 (1987).

It has been observed that 5-fluorouracil can enhance the activity of irinotecan. However, because 5-fluorouracil cannot be administered orally, the mode of administration for this combination therapy requires a more invasive form of administration such as by intravenous injection, and therefore typically requires administration by trained medical personnel.

It would be an advance in the art of treating tumors, especially colorectal cancerous tumors, if a therapy could be developed employing a potentiated form of irinotecan through the action of 5-fluorouracil in a convenient dosage form for oral administration.

### SUMMARY OF THE INVENTION

The present invention is directed to a dosage form(s) suitable for oral administration to a mammal for the treatment of tumors, especially colorectal tumors, that exhibits a synergistically enhanced effect in combination with irinotecan. In particular, there is provided in accordance with the present invention a dosage form(s) suitable for oral administration to a mammal having a tumor comprising an effective amount of each of tegafur, uracil, and folinic acid or a pharmaceutically acceptable salt thereof to a patient undergoing treatment with irinotecan, wherein said dosage form(s) is a potentiator of irinotecan. In a preferred form of the invention, tegafur and uracil are present in respective amounts sufficient for tegafur to effectively and efficiently convert to 5-fluorouracil. In a particularly preferred form of the invention tegafur and uracil are present in a molar ratio of about 1:4 (hereinafter referred to as “UFT”).

There is also disclosed a method of orally administering an anti-tumor effective amount of the combination of tegafur and uracil, preferably as UFT, and folinic acid or a pharmaceutically acceptable salt thereof to a mammal having a tumor who is undergoing irinotecan therapy.

The present invention further provides a method for the synergistic treatment of cancer, such as colorectal cancer, which comprises orally administering a synergistically effective amount of tegafur, uracil, and folinic acid or a pharmaceutically acceptable salt thereof, such as calcium folinate, to a mammal undergoing treatment with irinotecan.

### DETAILED DESCRIPTION OF THE INVENTION

The administration of the combination of tegafur and uracil in amounts sufficient to convert tegafur to 5-fluorouracil (preferably a molar ratio of about 1:4) can be administered orally. It was unexpectedly discovered that oral administration of this combination produced sufficient 5-fluorouracil that potentiation of irinotecan would take place despite the inability of 5-fluorouracil itself to be effectively administered orally. This was surprising because the combination of tegafur and uracil is not totally absorbed in the gut. Thus, it was unexpected that there would be a sufficient blood circulating concentration of 5-fluorouracil available to potentiate irinotecan.

The oral dosage form used in the present invention provides significant advantages over administering the combination by other modes of administration which are more invasive. In the treatment of tumors, a potential reduction in the cost of therapy because skilled medical personnel are not required to administer the drug and the psychological benefits afforded a patient by taking an oral medication provide significant benefits for patient care.

The dosage forms for all oral administration include tablets, powders, granules, and the like. Excipients and additives which may be used include, but are not limited to, lactose, sucrose, sodium chloride, glucose, urea, starch, calcium, kaolin, crystalline cellulose, salicylic acid, methylcellulose, glycerol, sodium alginate, arabic gum and the like. Conventional binders may be used such as glucose solutions, starch solutions, gelatine solutions, and the like. Disintegrators may be used including, but not limited to, dry starch, sodium alginate, agar powder, calcium carbonate, and the like. Absorbents which may be used include, but are not limited to, starch, lactose, kaolin, bentonite, and the like. Lubricants which may be used include, but are not limited to, purified talc, stearic acid salts, boric acid powder, polyethylene glycol and the like.

Tegafur, uracil, and folinic acid preferably provided as the calcium salt “calcium folinate” (leucovorin) are present in the oral dosage form(s) in an amount from about 1 to 70% by weight based on the total weight of the oral dosage form(s). The dosage of each active ingredient for administration on a daily basis is from about 0.1 to 100 mg/kg/day, preferably about 1 to 30 mg/kg/day for tegafur. The preferred dosage for uracil is from about 1 to 50 mg/kg/day. For UFT, i.e. the 1:4 combination of tegafur and uracil, the dosage is from about 200 to 500 mg/m²/day based on tegafur, preferably from about 250 to 300 mg/m²/day based on tegafur. Folinic acid or a pharmaceutically acceptable salt thereof may be administered in an amount from about 0.1 to 500 mg/kg/day, but preferably is administered as calcium folinate in a fixed dose of about 90 mg/day. The oral dosage form(s) may be administered in a single dose(s) or in divided doses typically up to 3 times a day. Irinotecan is typically administered in a non-oral mode of administration, typically intravenously. Based on body surface area, the dosage may range from about 100 to 400 mg/m²/day, preferably from about 200 to 300 mg/m²/day.

Those of ordinary skill in the art would have the knowledge to adjust the above stated dosage ranges for UFT, folinic acid or a pharmaceutically acceptable salt thereof, and irinotecan as needed based on body surface area and/or in the event of toxicity. In accordance with the present invention, the combination of tegafur and uracil (e.g. UFT) results in a sufficient amount of 5- fluorouracil available to potentiate irinotecan to improve the availability and potency of irinotecan in the treatment of tumors, especially colorectal tumors.

The following examples are exemplary of the claimed invention, but are not intended to limit the invention as encompassed by the full disclosure of the invention set forth herein.

### EXAMPLE 1

This study assessed the in vivo maximum tolerated dose (MTD), the side effect profile and the dose limited toxicity (DLT) of irinotecan combined with UFT (tegafur and uracil in a molar ratio of 1:4) plus calcium folinate (leucovorin).

An open label, phase I/II trial with escalating doses of UFT and irinotecan was conducted with a fixed dose of calcium folinate at 90 mg/day. Up to five cohorts of three to six patients comprised the test patient study. Entry criteria for the study included, but was not limited to, a histological or cytological confirmed metastatic colorectal carcinoma, no concurrent radiotherapy, no prior chemotherapy for metastatic disease, ECOG performance status of 0-2, no brain metastatic disease, and adequate hematological, renal and hepatic function.

The dosages for UFT, calcium folinate and irinotecan for each level are escalated as shown below in Table 1.

TABLE 1

| | | | Calcium | |
|---|---|---|---|---|
| | | | Folinate | |
| Cohort | Dose | UFT Dosage | Dosage | Irinotecan Dosage |
| No. | Level | (mg/m²/day) | (mg/day) | (mg/m²/day) |
| 1 | 0 | 250 | 90 | 200 |
| 2 | 1 | 250 | 90 | 250 |
| 3 | 2 | 250 | 90 | 300 |
| 4 | 3 | 300 | 90 | 300 |
| 5 | 4 | 300 | 90 | 300 |

The treatments were given on a three-week cycle until progressive disease or unacceptable toxicity occurred. UFT and calcium folinate were given orally on days 1-14 of each cycle; irinotecan was administered intravenously on day 1 (d1) of each cycle.

Six patients were entered at each dose level. After all patients had safely completed one cycle of treatment, the dose was escalated. The study continued to each progressive level until the maximum tolerated dose (MTD) was experienced. The MTD was defined as the dose level at which greater than ⅓ or 2/6 of the patients experienced a dose limiting toxicity (DLT) during the first cycle of treatment.

The cohort below the MTD was then expanded to 20 patients. The DLT was defined as follows:

a. Grade ¾ neutropenia complicated by fever greater than 38° C., I.V. antibiotics or grade ¾ diarrhea, or

b. Grade 4 thrombocytopenia prolonged or complicated by bleeding or requiring platelet transfusion, or

c. Grade ¾ neutropenia or thrombocytompenia for more than 7 days, or

d. Grade ¾ non-hematological toxicity with the exception of alopecia, nausea and vomiting, or

e. Grade greater than or equal to 2 renal, hepatic, cardiac or pulmonary toxicity or

f. A treatment delay of greater than two weeks prior to the start of the next cycle of treatment.

Patients qualified for the test protocol if they met the following criteria:

Histological or cytological confirmed advanced/metastatic colorectal carcinoma

Measurable disease (1 cm in at least one dimension)

Age >18 years

Either no prior chemotherapy or adjuvant chemotherapy for advanced or metastatic disease (>6 months prior to study entry) suitable for fluoropyrimidine or topoisomerase I inhibitor treatment

ECOG performance status 0-2, life expectancy >3 months

Serum creatinine 1.5×UNL

Serum bilirubin 1.5×UNL

Written informed consent

Patients were disqualified for the test protocol if they had bowel obstruction, any condition which would affect UFT and/or calcium folinate absorption, or prior radiotherapy unless palliative or for adjuvant therapy of rectal carcinoma.

The test patients were evaluated in the following manner:

Patients receiving at least one cycle of treatment were evaluated for toxicity.

Patients receiving at least 2 cycles of treatment or who developed rapid tumor progression or who died of progressive disease were evaluated for response. Also patients who discontinued treatment, or who died, due to a treatment related toxicity prior to response evaluation were considered evaluated for response.

Disease was assessed every 3 cycles and the response evaluated according to WHO criteria (Miller et al., 1981)

The study was closed after a total of 33 patients were registered for the study. Six patients were enrolled in each of Cohort Nos.1, 2, and 3. An additional patient was registered to Cohort No. 1, but was removed from the study before receiving treatment.

MTD was achieved at Cohort No. 3 (250 mg/m²/day UFT and 300 mg/m²/day 1 irinotecan). Accordingly, Cohort No. 2 was expanded to 20 patients. The pretreatment characteristics of the patients assigned to each cohort is shown in Table 2.

TABLE 2
Pre-treatment Characteristics

| | Cohort 1 | Cohort 2 | Cohort 3 | Total |
|---|---|---|---|---|
| | n = 7 | n = 20 | n = 6 | n = 33 (%) |
| Gender | | | | |
| Male | 3 | 11 | 4 | 18 (55) |
| Female | 4 | 9 | 2 | 15 (45) |
| Performances Status | | | | |
| 0 | 2 | 5 | 2 | 9 (27) |
| 1 | 5 | 12 | 3 | 20 (61) |
| 2 | 0 | 2 | 1 | 3 (9) |
| 3 | 0 | 1* | 0 | 1 (3) |
| Age | | | | |
| Median | 62 | 65 | 68 | 63 |
| Range | 35-74 | 47-80 | 19-63 | 19-80 |
| Primary Site | | | | |
| Colon | 4 | 7 | 5 | 16 (48) |
| Rectum | 3 | 9 | 1 | 13 (39) |
| Colorectal | 0 | 4 | 0 | 4 (12) |

*Not due to malignancy

The median number of cycles was 6. (range 1 -11)

The treatment was generally well tolerated. The most common grade ¾ toxicities were nausea, asthenia, diarrhea and neutropenia. The toxicity profile of the patients is shown in Table 3.

TABLE 3
CTC Worst grade 3/4 Adverse Events

| | Cohort 1 | Cohort 2 | Cohort 3 | Total |
|---|---|---|---|---|
| | n = 6 | n = 20 | n = 6 | n = 32 |
| Digestive system | | | | |
| Nausea | 2 | 3 | 2 | 7 |
| Vomiting | 0 | 2 | 1 | 3 |
| Diarrhea | 0 | 3 | 3 | 6 |
| Anorexia | 0 | 1 | 1 | 2 |
| Intestinal Obstruction | 0 | 1 | 1 | 2 |
| Constipation | 1 | 0 | 0 | 1 |
| Dyspepsia | 1 | 0 | 0 | 1 |
| Other non-hematologic | | | | |
| Asthenia | 1 | 6 | 2 | 9 |
| Headache | 1 | 0 | 0 | 1 |
| Pain | 1 | 1 | 2 | 4 |
| Myocardial Infarction | 0 | 1 | 0 | 1 |
| Infection | 0 | 1 | 0 | 1 |
| Respiratory system | 0 | 1 | 1 | 2 |
| Dyspnea | 0 | 1 | 1 | 2 |
| Somnolence | 0 | 1 | 0 | 1 |
| Epistaxis | 0 | 0 | 1 | 1 |
| Hematologic | | | | |
| Neutropenia | 0 | 8 | 2 | 10 |
| Febrile Neutropenia | 0 | 3 | 1 | 4 |

Dose limiting toxicities (DLT) were experienced by patients in Cohort Nos. 2 and 3 and included diarrhea and neutropenia as shown in Table 4.

TABLE 4
DLT (Exrerienced in first cycle treatment)

| Cohort 2 | Cohort 3 |
|---|---|
| (250 mg/m²/d UFT and | (250 mg/m²/d UFT and |
| 250 mg/m²/d1 irinotecan) | 300 mg/m²/d1 irinotecan) |
| Grade 4 diarrhea | Grade 3 diarrhea |
| | Grade 3 febrile neutropenia |
| | Grade 4 febrile neutropenia |

The response of the patients of Cohort Nos. 1-3 to treatment is shown in Table 5.

TABLE 5

| | Cohort 1 | Cohort 2 | Cohort 3 | Total (%) |
|---|---|---|---|---|
| EVALUATED | | | | |
| Complete response | 0 | 0 | 1 | 1 |
| (CR) | | | | |
| Partial response (PR) | 0 | 5 | 1 | 6 |
| Stable disease | 2 | 6 | 0 | 8 |
| Progressive disease | 0 | 1 | 1 | 2 |
| Response rate | | 35.7% | | 33.3% |
| (CR and PR) | | | | |
| Indeterminable | 0 | 2 | 2 | 4 |
| Total | 2 | 14 | 5 | 21 |

Of the 21 patients currently undergoing assessment the response to treatment was assessed according to criteria established by the World Health Organization (“WHO”) as described in Miller, A.B. et al., Cancer 11 (1981). A complete response in which the disappearance of all tumor lesions is confirmed by two independent observations at least four weeks apart, has been observed in one patient (4.8%). A partial response, in which there is generally a decrease of at least 50% in the size of tumor lesions was observed in 6 patients (28.6%). A stable disease situation was observed in 8 patients (38.1 %) where there was no change in the disease (i.e. a decrease in tumor size of less than 50% or an increase in tumor size of less than 25%) confirmed by two independent observations at least 4 weeks apart. A progressive disease situation was observed in 2 patients (9.5%). This category includes the appearance of any new, previously unidentified lesions or occurrence of malignant pleural effusion or ascites and/or an increase by at least 25% in the size of one or more measurable lesions.

Four patients exhibited early toxicity and were not therefore evaluated while one patient did not receive treatment. Eleven patients have not yet been assessed. Thus, the overall response rate for the study was 33.3% with a response rate of 35.7% at the dosage given to Cohort No. 2.

Based on the results shown in Table 5, the combination of UFT, calcium folinate (leucovorin) and irinotecan is well tolerated and shows anti-tumor activity.

What is claimed is:

1. A method of administering an anti-tumor effective amount of a combination of tegafur, uracil, and folinic acid or a pharmaceutically acceptable salt thereof, to a mammal undergoing treatment with irinotecan comprising administering to the mammal having a tumor said combination in oral dosage form(s).

2. The method of claim 1 wherein the amount of tegafur and uracil is sufficient to produce an effective amount of 5-fluorouracil sufficient to potentiate the activity of irinotecan.

3. The method of claim 1 wherein tegafur and uracil are present in a molar ratio of about 1:4, respectively.

4. A dosage form suitable for administration to a mammal having a tumor and undergoing treatment with irinotecan comprising an effective amount of each of tegafur, uracil, and folinic acid or a pharmaceutically acceptable salt thereof in oral dosage form.

5. The dosage form of claim 4 wherein the amount of tegafur and uracil is sufficient to produce an effective amount of 5-fluorouracil sufficient to potentiate the activity of irinotecan.

6. The dosage of claim 5 wherein tegafur and uracil are present in a molar ratio of about 1:4 sufficient to potentiate the activity of irinotecan.

7. A method for the synergistic treatment of cancer which comprises orally administering a synergistically effective amount of tegafur, uracil, and folinic acid or a pharmaceutically acceptable salt thereof to a mammal undergoing treatment with irinotecan.

8. The method according to claim 7 wherein the cancer is colorectal cancer.

9. The method according to claim 7 wherein tegafur and uracil are present in a molar ratio of about 1:4, respectively.

10. The method according to claim 7 wherein tegafur is orally administered at a dosage of about 1 to 30 mg/kg/day, uracil is orally administered at a dosage of about 1 to 50 mg/kg/day, and calcium folinate is orally administered at a fixed dosage of about 90 mg/day.

11. The method according to claim 7 wherein the mammal is treated with irinotecan at a dosage of about 100 to 400 mg/m²/day.

12. The method according to claim 7 wherein the mammal is treated with irinotecan at a dosage of about 200 to 300 mg/m²/day.

13. A method for the synergistic treatment of cancer which comprises orally administering a synergistically effective amount of UFT and folinic acid or a pharmaceutically acceptable salt thereof to a mammal undergoing treatment with irinotecan.

14. The method according to claim 13 wherein the cancer is colorectal cancer.

15. The method according to claim 13 wherein UFT is orally administered at a dosage of about 200 to 500 mg/m²/day based on tegafur, calcium folinate is orally administered at a fixed dosage of about 90 mg/day, and the mammal is treated with irinotecan at a dosage of about 100 to 400 mg/m²/day.

16. The method according to claim 13 wherein UFT is orally administered at a dosage of about 250 to 300 mg/m²/day based on tegafur, calcium folinate is orally administered at a fixed dosage of about 90 mg/day, and the mammal is treated with irinotecan at a dosage of about 200 to 300 mg/m²/day.
